(19) United States
(12) Patent Application Publication
(10) Pub. No.: US 2026/0335295 A1
(43) Pub. Date: Oct. 29, 2026

(54) PHARMACEUTICAL COMPOSITION COMPRISING HTRA ACTIVATOR AS ACTIVE INGREDIENT FOR PREVENTING OR TREATING DEGENERATIVE DISEASES, AND METHOD FOR SCREENING HTRA ACTIVITY MODULATOR

(71) Applicants: THE ASAN FOUNDATION, Seoul (KR); UNIVERSITY OF ULSAN FOUNDATION FOR INDUSTRY COOPERATION, Ulsan (KR); SUNGSHIN WOMEN'S UNIVERSITY R&DB FOUNDATION, Seoul (KR)

(72) Inventors: Min Ji KANG, Gyeonggi-do (KR); Jiyoun LEE, Gyeonggi-do (KR); Ji Yu LEE, Seoul (KR)

(73) Assignees: THE ASAN FOUNDATION, Seoul (KR); UNIVERSITY OF ULSAN FOUNDATION FOR INDUSTRY COOPERATION, Ulsan (KR); SUNGSHIN WOMEN'S UNIVERSITY R&DB FOUNDATION, Seoul (KR)

(21) Appl. No.: 19/548,425

(22) Filed: Feb. 24, 2026

Related U.S. Application Data
(63) Continuation-in-part of application No. PCT/KR2024/012593, filed on Aug. 23, 2024.

(30) Foreign Application Priority Data
Aug. 25, 2023 (KR) 10-2023-0111652

(51) Int. Cl. A61K 31/426; A61P 27/02; C07D 277/30; G01N 33/50

(52) U.S. Cl. CPC A61K 31/426; A61P 27/02; C07D 277/30; G01N 33/502

(57) ABSTRACT

A compound represented by Formula 1, a stereoisomer thereof, a solvate thereof, a hydrate thereof, or a pharmaceutically acceptable salt thereof according to one aspect can be utilized as a preventive or therapeutic agent and the like for degenerative diseases, such as macular degeneration (AMD), cerebral amyloid angiopathy, hereditary stroke (CADASIL), and Alzheimer's disease (AD), in which HTRA1 activity deficiency is a factor in the aggravation of the diseases, and can be utilized as an adjuvant for efficiently and accurately performing screening of an HTRA activity modulator without temperature changes or the addition of substrates.

### TECHNICAL FIELD

The present disclosure relates to a pharmaceutical composition for preventing or treating a degenerative disease, including a high temperature requirement A (HTRA) serine protease activator as an active ingredient, and a method of screening an HTRA activity modulator.

### BACKGROUND ART

High temperature requirement A (HTRA) serine protease is involved in protein quality control and cellular stress responses in various organisms including humans, fungi, plants, mammals, and bacteria. Specifically, HTRA is known to associated with cellular stress responses and protein quality control of mammalian cells and microorganisms.

HTRA consists of a protease domain and a PDZ domain, and this multi-binding domain complex is characterized by being oligomerized according to their subtypes and functional states, HTRA plays an important role in alleviating protein-folding stress and regulating cellular responses related to stress by functioning as a chaperone as well as a protease.

Since HTRA is ubiquitously expressed in a wide range of organisms, it is associated with the pathogenesis of many diseases. For example, it has been reported that HTRA1 degrades amyloid precursor protein (APP), amyloid β aggregates, and tau fibrils related to Alzheimer's disease, and decreased HTRA2 activity is known to be associated with Parkinson's disease. Additionally, changes in the activity of human HTRA are associated with art itis, cancer, cerebral small vessel disease, age-related macular degeneration, and neurodegenerative diseases (Literature [Journal of Visualized Experiments Volume 2016, Issue 113, Article number e54136]).

The activity of the HTRA is triggered by two different mechanisms: first, by the binding of an allosteric effector to the PDZ domain that induces cooperative remodeling into an active conformation; and second, by the binding of a peptide substrate to the active site that promotes substrate-induced oligomerization. Through the HTRA activation mechanism, an HTRA activator having good cell permeability may be used for the treatment of degenerative diseases caused by decreased HTRA activity:

However, the process of activating and converting the functional state of HTRA has not been completely elucidated, and research on HTRA-specific small-molecule activity modulators is currently insufficient.

The activity of the HTRA enzyme is maintained at a low level in the absence of an activator; thus, conventional methods have used a method of adding an excess amount of a substrate or promoting a reaction with a substrate at 45 °C through heating to perform an enzyme activity assay. Howe activity is greatly affected by temperature changes, where each e acts most effectively at a specific temperature, and the activity decreases if the temperature is higher or lower than the specific temperature (Campbell Biology 4th ed. FIG. 6.13, p101).

Accordingly, there is a need to develop a pharmaceutical composition for preventing or treating a degenerative disease using an HTRA activator and a method of efficiently screening an HTRA activity modulator at room temperature.

### DISCLOSURE OF INVENTION

### Technical Problem

One aspect is to provide a compound represented by Formula 1, a stereoisomer thereof, a solvate thereof, a hydrate thereof, or a pharmaceutically acceptable salt thereof.

Another aspect is to provide a pharmaceutical composition for preventing or treating a degenerative disease, including the compound of Formula 1, a stereoisomer thereof, a solvate thereof, a hydrate thereof, or a pharmaceutically acceptable salt thereof as an active ingredient.

Another aspect is to provide a health functional food for preventing or improving a degenerative disease, including the compound of Formula 1, a stereoisomer thereof, a solvate thereof, a hydrate thereof, or a pharmaceutically acceptable salt thereof as an active ingredient.

Another aspect is to provide a reagent composition for HTRA activation, including the compound of Formula 1, a stereoisomer thereof, a solvate thereof, a hydrate thereof, or a pharmaceutically acceptable salt thereof as an active ingredient.

Another aspect is to provide a method of screening a high temperature requirement A (HTRA) activity modulator, the method including: treating a sample with the compound of Formula 1, a stereoisomer thereof, a solvate thereof, a hydrate thereof, or a pharmaceutically acceptable salt thereof in vitro; treating the sample treated with the compound of Formula 1, the stereoisomer thereof, the solvate thereof, the hydrate thereof, or the pharmaceutically acceptable salt thereof with a candidate substance; identifying an activity level of HTRA; and comparing the activity level of HTRA with an activity level of HTRA of a sample not treated with the candidate substance.

Another aspect is to provide use of a composition for preventing or treating a degenerative disease, the composition including the compound of Formula 1, a stereoisomer thereof, a solvate thereof, a hydrate thereof, or a pharmaceutically acceptable salt thereof as an active ingredient.

Another aspect is to provide a method of preventing or treating a degenerative disease, the method including administering a composition including an effective amount of the compound of Formula 1, a stereoisomer thereof, a solvate thereof, a hydrate thereof, or a pharmaceutically acceptable salt thereof as an active ingredient to a subject in need thereof.

Another aspect is to provide use of a composition for use in the manufacture of a medicament for preventing or treating a degenerative disease, the composition including the compound of Formula 1, a stereoisomer thereof, a solvate thereof, a hydrate thereof, or a pharmaceutically acceptable salt thereof as an active ingredient.

### Solution to Problem

In the present disclosure, when a certain part “includes” a certain component, this means that the part may further include other components rather than excluding other components unless otherwise stated.

One aspect provides a compound represented by Formula 1, an isomer thereof, a solvate thereof, a hydrate thereof, or a pharmaceutically acceptable salt thereof.

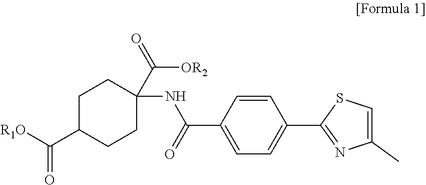

In Formula 1, R₁ and R₂ are independently hydrogen, C₁-C₁₀ alkyl, C₂-C₁₀ alkenyl, or C₂-C₁₀ alkynyl.

In an embodiment, the compound may be one wherein R₁ and R₂ are independently hydrogen or C₁-C₄ alkyl, or a stereoisomer, solvate, hydrate, or pharmaceutically acceptable salt thereof.

In an embodiment, the compound may be one wherein R₁ and R₂ are independently hydrogen or methyl, or a stereoisomer, solvate, hydrate, or pharmaceutically acceptable salt thereof.

One aspect provides a pharmaceutical composition for preventing or treating a degenerative disease, including a compound of Formula 1, a stereoisomer thereof, a solvate thereof, a hydrate thereof, or a pharmaceutically acceptable salt thereof as an active ingredient.

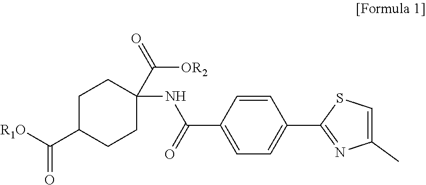

In Formula 1,
R₁ and R₂ are independently hydrogen, C₁-C₁₀ alkyl, C₂-C₁₀ alkenyl, or C₂-C₁₀ alkynyl.

In the present disclosure, the term “alkyl” refers to a hydrocarbon having primary, secondary, tertiary, and/or quaternary carbon atoms, and may be linear, branched, or a combination thereof.

In an embodiment, the alkyl group may have 1 to 10 carbon atoms (i.e., C₁-C₁₀ alkyl), 1 to 4 carbon atoms (i.e., C₁-C₄ alkyl), or a methyl group.

For example, suitable alkyl groups may include, but are not limited to: methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃) CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃). 3-methyl-2-butyl (—CH(CH₃) CH(CH₃)₂), 3-methyl-1-butyl (—CH₂CH₂CH(CH₃))), 2-methyl-1-butyl (—CH₂CH(CH₃)CH₂CH₃), 1-hexyl (—CH₂CH₂CH₂CH₂CH₂CH₃), 2-hexyl (—CH(CH₃) CH₂CH₂CH₂CH₃), 3-hexyl (—CH(CH₂CH₃)(CH₂CH₂CH₃), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)₂(CH(CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), and octyl (—(CH₂)7CH₃).

In the present disclosure, the term “alkenyl” refers to a substituent derived from a linear or branched unsaturated hydrocarbon having 2 to 10 carbon atoms and having at least one carbon-carbon double bond. For example, the alkenyl may be vinyl, allyl, isopropenyl, 2-butenyl, or the like, but is not limited thereto.

In the present disclosure, the term “alkynyl” refers to a substituent derived from a linear or branched unsaturated hydrocarbon having 2 to 10 carbon atoms and having at least one carbon-carbon triple bond. For example, the alkynyl may be ethynyl or 2-propynyl, but is not limited thereto.

In the present disclosure, the term “isomer” refers to a compound of the present disclosure or a salt thereof having the same chemical formula or molecular formula but being structurally or stereochemically different. Such isomers include structural isomers such as tautomers caused by a change in the position of a hydrogen atom and stereoisomers, and the stereoisomers include all of R or S isomers (enantiomers) having an asymmetric carbon center, geometric isomers (trans, cis), and the like.

In the present disclosure, isomers of the compound represented by Formula 1 and mixtures thereof are also included in the scope of the present disclosure.

In the present disclosure, the term “hydrate” refers to a compound of the present disclosure or a salt thereof containing a stoichiometric or non-stoichiometric amount of water bonded by a non-covalent intermolecular force. Specifically, the hydrate of the compound represented by Formula 1 of the present disclosure may include a stoichiometric or non-stoichiometric amount of water bonded by a non-covalent intermolecular force. For example, the hydrate may contain 1 equivalent or more, and preferably, 1 to 5 equivalents of water.

In an embodiment, the hydrate may be prepared by crystallizing the compound represented by Formula 1 of the present disclosure, an isomer thereof, or a pharmaceutically acceptable salt thereof from water or a solvent containing water.

In the present disclosure, the term “solvate” refers to a compound of the present disclosure or a salt thereof containing a stoichiometric or non-stoichiometric amount of a solvent bonded by a non-covalent intermolecular force. Preferred solvents therefor include solvents that are volatile, non-toxic, and/or suitable for administration to humans.

In the present disclosure, the term “pharmaceutically acceptable salt” refers to a salt that is pharmaceutically acceptable and maintains the desired pharmacological activity of a parent compound. The salt is not particularly limited as long as it is pharmaceutically acceptable. For example, an acid addition salt formed by a pharmaceutically acceptable free acid may be used as the pharmaceutically acceptable salt. Specifically, the acid addition salt may be obtained from inorganic acids such as hydrochloric acid, nitric acid, phosphoric acid, sulfuric acid, hydrobromic acid, hydroiodic acid, nitrous acid, and phosphorous acid; non-toxic organic acids such as aliphatic mono- and di-carboxylates, phenyl-substituted alkanoates, hydroxy alkanoates and alkanedioates, aromatic acids, and aliphatic and aromatic sulfonic acids; and organic acids such as trifluoroacetic acid, acetate, benzoic acid, citric acid, lactic acid, maleic acid, gluconic acid, methanesulfonic acid, 4-toluenesulfonic acid, tartaric acid, and fumaric acid. Types of the pharmaceutically non-toxic salts include, but are not limited to, sulfate, pyrosulfate, bisulfate, sulfite, bisulfite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate, mandelate, and the like. The pharmaceutically acceptable salt according to the present disclosure may be prepared by a conventional method.

In the present disclosure, the term “degenerative disease” refers to a disease in which functional and structural damage to a tissue or organ gradually worsens over time.

Specifically, the degenerative disease refers to a degenerative disease that occurs according to an activity level of high temperature requirement A (HTRA).

In an embodiment, the degenerative disease may be age-related macular degeneration (AMD). The age-related macular degeneration (AMD) may be one selected from the group consisting of wet macular degeneration, dry macular degeneration, geographic atrophy, and diabetic retinopathy, but is not limited thereto.

In an embodiment, the degenerative disease may be a neurodegenerative disease. The neurodegenerative disease may be one selected from the group consisting of dementia, Alzheimer's disease (AD), Parkinson's disease, Huntington's disease, cerebral amyloid angiopathy, cerebral amyloid vasculopathy, amyloid-related stroke, systemic amyloidosis, Dutch-type amyloidosis, hereditary stroke (CADASIL), amyotrophic lateral sclerosis, spinocerebellar atrophy, Friedrich's ataxia, Machado-Joseph's disease, Lewy body dementia, dystonia, progressive supranuclear palsy, and frontotemporal dementia, but is not limited thereto.

The HTRA is a type of serine protease involved in the composition of an extracellular matrix, cell proliferation, and aging. An activity level of HTRA is associated with various diseases including Duchenne muscular dystrophy (Bakay et al. 2002, Neuromuscul. Disord 12:125-141), arthritis, such as osteoarthritis (Grau et al. 2006, JBC 281:6124-6129), cancer, familial ischemic cerebral small-vessel disease, and age-related. macular degeneration, as well as Parkinson's disease and Alzheimer's disease. For example, HTRA1 inhibits signaling by the TGF β family of growth factors (Oka et al., 2004, Development 131:1041-1053). HTRA1 is capable of cleaving amyloid precursor protein (APP), and HTRA1 inhibitors cause accumulation of Aβ peptides in cultured cells. Accordingly, decreased activity of HTRA1 is associated with Alzheimer's disease (Grau et al., 2005, Proc. Nat. Acad. Sci. USA. 102:6021-6026).

The term “prevention” as used herein may include, without limitation, any action capable of blocking symptoms caused by a degenerative disease or inhibiting or delaying the progression thereof using the composition of the present disclosure. The term “treatment” as used herein refers to a series of activities performed for the alleviation and improvement of a target disease.

In an embodiment, the pharmaceutical composition may activate high temperature requirement A (HTRA).

The term “high temperature requirement A (HTRA)” as used herein refers to one of the serine proteases that play a role in resolving protein-folding problems and mediating the degradation of damaged proteins. HTRA is involved in protein quality control and cellular stress responses in various organisms including humans, fungi, plants, mammals, and bacteria.

In the case of microorganisms, high temperature requirement A (HTRA) is known to be found in species with high pathogenicity and high antibiotic resistance, such as Helicobacter pylori species or Chlamydia species, and is known to be involved in invasive action against host cells and involved in the survival of microorganisms in heat stress or acid stress environments.

In the case of humans, four isoforms of HTRA, specifically HTRA1, HTRA2, HTRA3, and HTRA4, exist. HTRA1 is known to be located in the cell membrane, cytoplasm, and cell nucleus, and HTRA2 is known to be located inside the mitochondria. The human high temperature requirement A (HTRA) family is a universally expressed PDZ-protease involved in maintaining protein homeostasis in an extracellular compartment by combining dual functions of a protease and a chaperone.

In an embodiment, the HTRA may be at least one selected from HTRA1 and HTRA2.

The pharmaceutical composition according to the present disclosure may further include a suitable carrier, excipient, or diluent commonly used in the manufacture of pharmaceutical compositions.

The carrier, excipient, or diluent usable in the present disclosure include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, or mineral oil.

The pharmaceutical composition according to the present disclosure may be formulated and used in the form of oral dosage forms such as powders, granules, tablets, capsules, suspensions, emulsions, syrups, and aerosols, external preparations, suppositories, and sterile injection solutions, each according to a conventional method. In the case of formulation, the composition is prepared using a diluent or excipient such as a commonly used filler, extender, binder, wetting agent, disintegrant, or surfactant.

Solid preparations for oral administration include tablets, pills, powders, granules, capsules, and the like, and these solid preparations may be prepared by mixing the compound with at least one or more excipients, for example, starch, calcium carbonate, sucrose, lactose, gelatin, or the like. In addition, lubricants such as magnesium stearate and talc are used in addition to simple excipients. Liquid preparations for oral administration include suspensions, oal solutions, emulsions, syrups, and the like, and in addition to water and liquid paraffin, which are commonly used simple diluents, various excipients such as wetting agents, sweeteners, fragrances, and preservatives may be included.

Preparations for parenteral administration include sterile aqueous solutions, non-aqueous solvents, suspensions, emulsions, lyophilized preparations, and suppositories. As the non-aqueous solvents and suspensions, propylene glycol, polyethylene glycol, vegetable oils such as olive oil, and injectable esters such as ethyl oleate may be used.

As a base of the suppositories, Witepsol, macrogol, Tween 61, cacao butter, laurin butter, glycerogelatin, and the like may be used.

In addition, the dosage of the pharmaceutical composition according to the present disclosure may increase or decrease depending on a route of administration, the severity of a disease, gender, body weight, age, and the like, and may be appropriately selected by a skilled medical professional. Therefore, the dosage does not limit the scope of the present disclosure in any way.

The pharmaceutical composition may be administered to mammals such as rats, mice, livestock, and humans through various routes. For example, the pharmaceutical composition may be administered by oral, rectal, or intravenous, intramuscular, subcutaneous, intrabronchial inhalation, intrauterine, intrathecal, of intracerebroventricular injection, but is not limited thereto.

Another aspect provides a health functional food for preventing or improving a degenerative disease, including a compound of Formula 1, a stereoisomer thereof, a solvate thereof, a hydrate thereof, or a pharmaceutically acceptable salt thereof as an active ingredient.

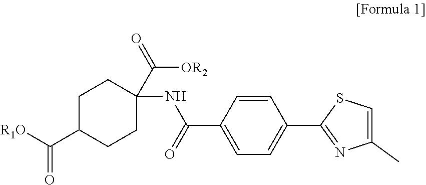

In Formula 1;
R₁ and R₂ are independently hydrogen, C₁-C₁₀ alkyl, C₂-C₁₀ alkenyl, or C₂-C₂ alkynyl.

The term “improvement” as used herein refers to alleviation or relief of symptoms of a degenerative disease by administering, ingesting, or applying the pharmaceutical composition or food composition of the present disclosure to an individual suffering from the degenerative disease.

The term “health functional food” as used herein refers to a food manufactured and processed using raw materials or ingredients having functionality useful for the human body according to the Health Functional Food Act, and the term “functionality” as used herein refers to ingestion for the purpose of obtaining effects useful for health purposes such as regulating nutrients or physiological actions with respect to the structure and function of the human body. Specifically, the health functional food refers to a food manufactured and processed in the form of tablets, capsules, powders, granules, liquids, pills, or the like using raw materials or ingredients having functionality useful for the human body. Types of the food are not particularly limited and include all health functional foods in a conventional sense.

The health functional food of the present disclosure may be manufactured by a method commonly used in the art, and during the manufacture, the food may be manufactured by adding raw materials and ingredients commonly added in the art. In addition, a dosage form of the health functional food may be manufactured without limitation as long as the dosage form is recognized as a health functional food. Specifically, the health functional food may be provided in the form of a powder, a granule, a tablet, a capsule, a syrup, or a beverage.

The health functional food is used together with other foods or food additives in addition to the compound represented by Formula 1, a stereoisomer thereof, a solvate thereof, a hydrate thereof, or a pharmaceutically acceptable salt thereof, which is an active ingredient, and may be appropriately used according to a conventional method. A mixed amount of the active ingredient may be appropriately determined according to a purpose of use thereof, for example, prevention, health, or therapeutic treatment.

The matters mentioned in the pharmaceutical composition and the health functional food of the present disclosure apply equally unless they contradict each other.

Another aspect provides a method of preventing or treating a degenerative disease, including administering the compound of Formula 1, a stereoisomer thereof, a solvate thereof, a hydrate thereof, or a pharmaceutically acceptable salt thereof to a subject in need thereof.

Another aspect provides use of the compound of Formula 1, a stereoisomer thereof, a solvate thereof, a hydrate thereof, or a pharmaceutically acceptable salt thereof for use in the manufacture of a medicament for preventing or treating a degenerative disease.

In the method or the use, the detailed description regarding the aforementioned pharmaceutical composition may be applied.

Another aspect provides a reagent composition for HTRA activation, including the compound of Formula 1, a stereoisomer thereof, a solvate thereof, a hydrate thereof, or an acceptable salt thereof as an active ingredient.

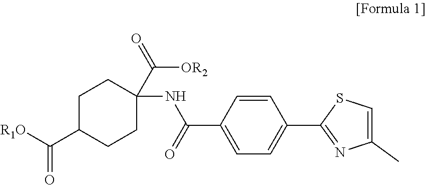

In Formula 1,
R₁ and R₂ are independently hydrogen, C₁-C₁₀ alkyl, C₂-C₁₀ alkenyl, or C₂-C₁₀ alkynyl.

In an embodiment, the reagent composition may include the compound of Formula 1, a stereoisomer thereof, a solvate thereof, a hydrate thereof, or a pharmaceutically acceptable salt thereof at a concentration of 10 μM to 100 μM. For example, the reagent composition may include the compound of Formula 1, a stereoisomer thereof, a solvate thereof, a hydrate thereof, or an acceptable salt thereof at a concentration of 10 μM to 20 μM, 10 μM to 30 μM, 10μ M to 40 μM, 10 μM to 50 μM, 10 μM to 60 μM, 10 μM to 70 μM, 10 μM to 80 μM, 10 μM to 90 μM, 10 μM to 100 μM, 20 μM to 30 μM, 20 μM to 40 μM, 20 μM to 50 μM, 20 μM to 60 μM, 20 μM to 70 μM, 20 μM to 80 μM, 20 μM to 90 μM, 20 μM to 100 μM, 30 μM to 40 μM, 30 μM to 50 μM, 30 μM to 60 μM, 30 μM to 70 μM, 30 μM to 80 μM, 30 μM to 90 μM, 30 μM to 100 μM, 40 μM to 50 μM, 40 μM to 60 μM, 40 μM to 70μ, 40 μM to 80 μM, 40 μM to 90 μM, 40 μM to 100 μM, 50 μM to 60 μM, 50 μM to 70 μM, 50 μM to 80 μM, 50 μM to 90 μM, 50 μM to 100 μM, 60 μM to 70 μM, 60 μM to 80 μM, 60 μM to 90 μM, 60 μM to 100 μM, 70 μM to 80 μM, 70 μM to 90 μM, 70 μM to 100 μM, 80 μM to 90 μM, 80 μM to 100 μM, or 90 μM to 100 μM, but is not limited thereto.

The enzyme activity assay refers to measuring the activity level of an enzyme by a method such as measuring a reduction rate of a substrate of a specific enzyme or an increase rate of a product thereof.

The reagent composition for HTRA activation including the compound of Formula 1, a stereoisomer thereof, a solvate thereof, a hydrate thereof, or an acceptable salt thereof enables the HTRA enzyme activity assay without temperature changes or the addition of substrates by a method of modifying the conformation of the enzyme through binding to an allosteric site of the HTRA enzyme.

Another aspect provides a method of screening a high temperature requirement A (HTRA) activity modulator, the method including: activating HTRA by treating a sample including HTRA with a compound of Formula 1 below, a stereoisomer thereof, a solvate thereof, a hydrate thereof, or an acceptable salt thereof in vitro; treating the sample including the activated HTRA with a candidate substance; and comparing an HTRA activity level of the sample treated with the candidate substance with an HTRA activity level of a sample not treated with the candidate substance,

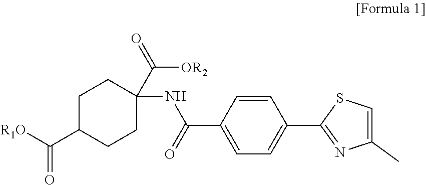

In Formula 1,
R₁ and R₂ are independently hydrogen, C₁-C₁₀ alkyl, C₂-C₁₀ alkenyl, or C₂-C₁₀ alkynyl.

In an embodiment, the comparing may include selecting a candidate substance that has changed an HTRA activity level as an HTRA modulator.

In an embodiment, the screening method may be characterized by selecting the candidate substance as an HTRA activator when an HTRA activity level of a sample treated with the candidate substance is increased compared to an HTRA activity level of a sample not treated with the candidate substance.

In an embodiment, the screening method may be characterized by selecting the candidate substance as an HTRA inhibitor when an HTRA activity level of a sample treated with the candidate substance is decreased compared to an HTRA activity level of a sample not treated with the candidate substance.

The identifying of the HTRA activity level may employ an HTRA activity-based peptide probe for HTRA detection but is not limited thereto. Specifically, the HTRA activity-based peptide probe for HTRA detection refers to being characterized by detecting human HTRA1, human HTRA2, and microorganism-derived HTRA on an activity basis.

In an embodiment, the sample may be one selected from the group consisting of a cell, a tissue, blood, serum, urine, and saliva.

Another aspect provides use of a composition for preventing or treating a degenerative disease, the composition including a compound of Formula 1 below, a stereoisomer thereof, a solvate thereof, a hydrate thereof, or a pharmaceutically acceptable salt thereof as an active ingredient.

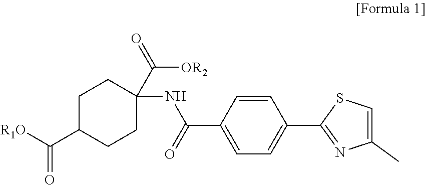

In Formula 1, R₁ and R₂ are independently hydrogen, C₁-C₁₀ alkyl, C₂-C₁₀ alkenyl, or C₂-C₁₀ alkynyl.

Another aspect provides a method of preventing or treating a degenerative disease, the method including administering a composition including an effective amount of the compound of Formula 1, a stereoisomer thereof, a solvate thereof, a hydrate thereof, or a pharmaceutically acceptable salt thereof as an active ingredient to a subject in need thereof. In Formula 1, R₁ and R₂ are independently hydrogen, C₁-C₁₀ alkyl, C₂-C₁₀ alkenyl, or C₂-C₁₀ alkynyl.

Another aspect provides use of a composition for use in the manufacture of a medicament for preventing or treating a degenerative disease, the composition including the compound of Formula 1, a stereoisomer thereof, a solvate thereof, a hydrate thereof, or a pharmaceutically acceptable salt thereof as an active ingredient. In Formula 1, R₁ and R₂ are independently hydrogen, C₁-C₁₀ alkyl, C₂-C₁₀ alkenyl, or C₂-C₁₀ alkynyl.

The terms, methods, and the like described for the present disclosure are identically applied among the respective aspects.

### Advantageous Effects of Invention

A compound represented by Formula 1, a stereoisomer thereof, a solvate thereof, a hydrate thereof, or a pharmaceutically acceptable salt thereof according to one aspect can be utilized as a preventive or therapeutic agent for degenerative diseases, such as macular degeneration (AMD), cerebral amyloid angiopathy, hereditary stroke (CADASIL), and Alzheimer's disease (AD), in which HTRA1 activity deficiency is a factor in the aggravation of the diseases, and can be utilized as an adjuvant for efficiently and accurately performing screening of an HTRA activity modulator without temperature changes or the addition of substrates.

### BRIEF DESCRIPTION OF DRAWINGS

FIG. 1 is a graph showing results of an HTRA1 activity assay of compounds E01 to E12 obtained through Atomwise.

FIGS. 2A to 2D are is a graphs showing changes in HTRA1 activity according to a dose of E09: FIG. 2A is a graph showing a change in HTRA1 activity when a dose of E09 is 1 nM to 1 μM; FIG. 2B is a graph showing a change in HTRA1 activity when a dose of E09 is 1 nM to 10 μM; FIG. 2C is a graph showing a change in HTRA1 activity when a dose of E09 is 0 μM to 100 μM; and FIG. 2D is a graph showing HTRA1 activity when E09 is added (final concentration of 10 μM) and when E09 is not added.

FIGS. 3A to 3B are is a graphs showing changes in HTRA2 activity according to a dose of E09: FIG. 3A is a graph showing a change in HTRA2 activity when a dose of E09 is 1 nM to 1 μM; and FIG. 3B is a graph showing a change in HTRA2 activity when a dose of E09 is 1 nM to 10 μM.

FIG. 4 is a photograph of HTRA1 activity observed with a fluorescence microscope after treating HeLa cells with an HTRA1 probe (DS-1-94, final concentration of 5 μM) and E09 (HTRA activator, final concentration of 10 μM).

FIG. 5 is a graph showing HTRA1 enzyme activity measured using casein, known as a substrate of HTE enzymes, or PDZ-opt (GQYYFV), known as a peptide that activates HTRA enzymes, and E09.

FIG. 6 is a schematic diagram showing a construction process of a dry macular degeneration cell model by NaIO₃ treatment and experimental conditions for each treatment group.

FIG. 7 is a set of images identifying changes in a cobblestone appearance structure according to NaIO₃ treatment through clusterin and ZO-1 staining (FIGS. 7A-D) and cell death confirmed by a TUNEL assay (FIGS. 7E-H).

FIG. 8 is a schematic diagram illustrating a construction process of a three-dimensional long-term culture macular degeneration cell model and experimental conditions for each treatment group.

FIG. 9 is a set of images identifying expression levels of ApoE and Cathepsin D confirmed by immunostaining (FIGS. 9A-C), changes in a cell barrier structure according to clusterin and ZO-1 staining (FIGS. 9D-F), results of observing microvilli formation (FIGS. 9G-I), and results of holotomography analysis (FIGS. 9I-L).

FIG. 10 is a schematic diagram illustrating the process for constructing a mouse macular degeneration pathology model induced by administration of NaIO₃.

FIG. 11 shows results of comparative analysis on retinal structure protective effects of compound E09 and an HtrA1 inhibitor in a mouse macular degeneration pathology model induced by NaIO₃ administration:

FIG. 11A is a set of histological images observing morphological changes in a retinal pigment epithelium (RPE) layer, photoreceptor outer segments (POS), and an outer nuclear layer (ONL) by Hematoxylin & Eosin (H&E) staining of retinal tissues of a control group (DMSO), an E09-treated group, and an HtrA1 inhibitor-treated group after administration of NaIO₃; and FIG. 11B is statistical graphs comparing measurements of total retina thickness and ONL thickness to quantify the degree of retinal damage in each treatment group.

FIG. 12 is a graph showing the results of GSEA (Gene Set Enrichment Analysis) performed after transcriptome analysis following treatment with Compound E09 or an HTRA1 inhibitor in a pathological model of macular degeneration (AMD).

### MODE FOR THE INVENTION

The present disclosure is described in more detail below through examples. However, these examples are provided for illustrative purposes only and are not intended to limit the scope of the present disclosure.

### Example 1. Identification of HTRA Activators

Activity screening was performed for 72 types of compounds obtained through Atomwise, an artificial intelligence-based compound design company. The 72 types of compounds were designed utilizing the active site structure of DegP (Degradation of periplasmic proteins), which is a bacterial homolog of the HTRA family.

HTRA activity was identified through high-throughput screening using H2-Opt, a commercially available fluorogenic substrate.

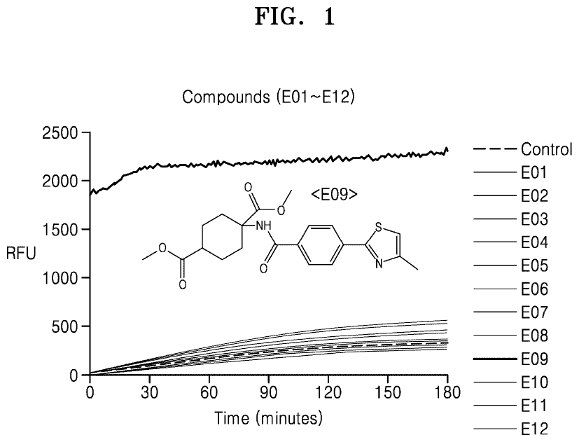

Specifically, purified recombinant HTRA1 protein (50 ng) and a compound (final concentration of 10 μM) were added to 80 μL of an assay buffer in each well of a 96-well plate and were allowed to stand at room temperature for 30 minutes. Immediately after 20 μL of an H2-OPT solution (final concentration of 6.25 μM) was added thereto and mixed, fluorescence signals generated therefrom were measured using an MS microplate reader device manufactured by Molecular Devices. Upon measurement of the fluorescence signals in kinetic mode, excitation and emission were set to 328 nm and 393 nm, respectively, and changes in the fluorescence signals were measured at 37 °C for 3 hours at intervals of 30 seconds. Results of measuring the HTRA activity through high-throughput screening are shown in FIG. 1.

FIG. 1 is a graph showing the results of an HTRA1 activity assay for the compounds E01 to E12 obtained through Atomwise.

As shown in FIG. 1, among the compounds E01 to E12, the compound E09 was found to significantly activate HTRA1. The compound E09 was confirmed to exhibit excellent activation ability in both HTRA1 and HTRA2.

For reference, the structure of the compound E09 is represented by Formula 2 below.

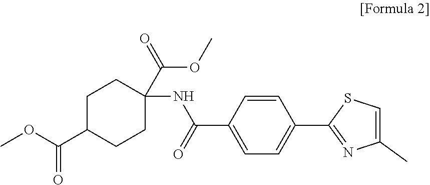

The compound E09 was purchased from Enamine (NI, USA) (Catalog No. Z1571762517) and used.

The IUPAC name of compound E09 is dimethyl 1-(4-(4-methylthiazol-2-yl)benzamido)cyclohexane-1,4-dicarboxylate.

### Example 2. Measurement of Dose-Dependent Activity of HTRA1 and HTRA2 by E09

Changes in activity according to a dose of E09 (dose dependency of HTRA1 and HTRA2 activity) were identified for HTRA1 and HTRA2, respectively.

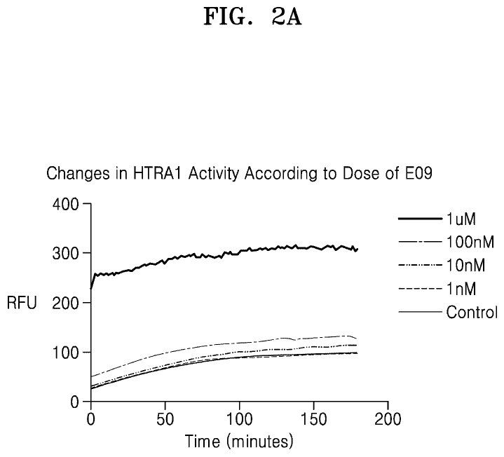

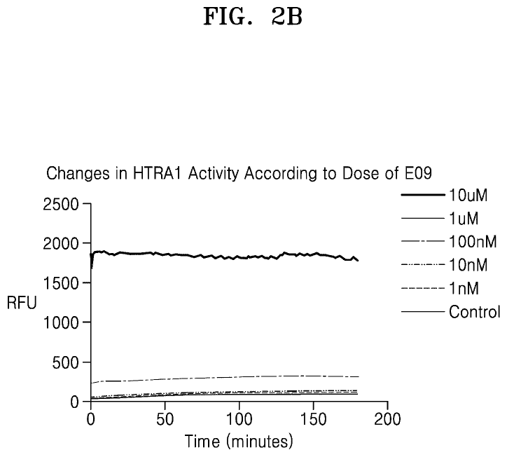

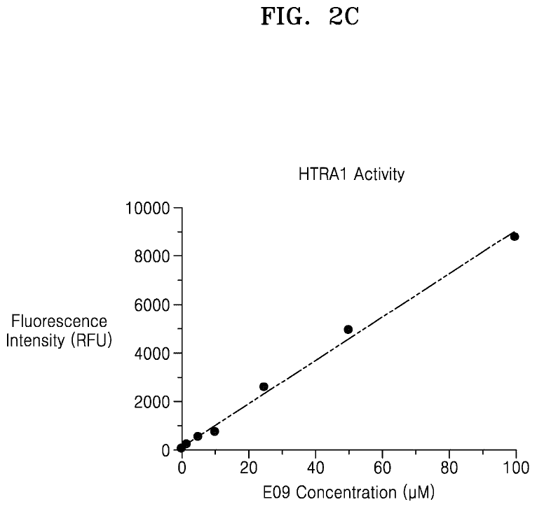

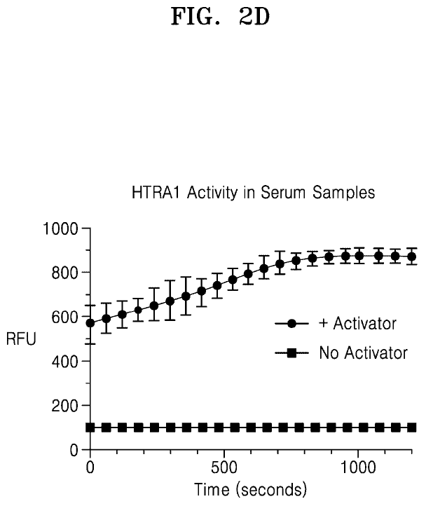

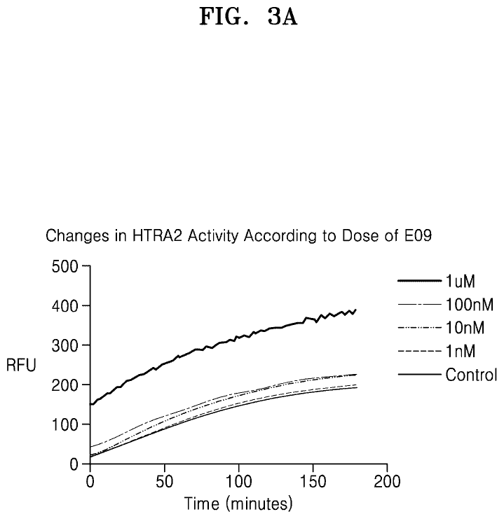

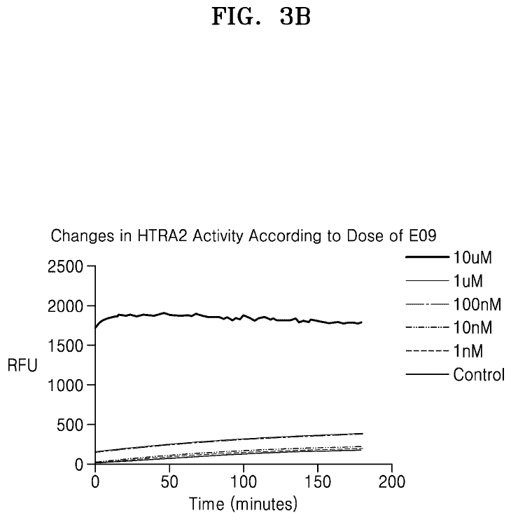

Specifically, different concentrations of E09 (0.01, 0.1, 1, 10, and 100 (M) w ere added to purified recombinant HTRA1 and HTRA2, and activities of HTRA1 and HTRA2 were measured in the same manner as the high-throughput screening of Example 1. Fluorescence signals at the end of measurement (3 hours) were measured for each concentration. Graphs obtained by measuring the fluorescence signals of HTRA1 and HTRA2 are shown in FIGS. 2 and 3.

FIG. 2 is a graph showing changes in HTRA1 activity according to a dose of E09;

FIG. 2A is a graph showing a change in HTRA1 activity when a dose of E09 is 1 nM to 1 μM; FIG. 2B is a graph showing a change in HTRA1 activity when a dose of E09 is 1 nM to 10 μM; FIG. 2C is a graph showing a change in HTRA1 activity when a dose of E09 is 0 μM to 100 μM; and FIG. 2D is a graph showing HTRA1 activity when E09 is added (final concentration of 10 μM) and when E09 is not added.

FIG. 3 is a graph showing changes in HTRA2 activity according to a dose of E09: FIG. 3A is a graph showing a change in HTRA2 activity when a dose of E09 is 1 nM to 1 μM; and FIG. 3B is a graph showing a change in HTRA2 activity when a dose of E09 is 1 nM to 10 μM.

As shown in FIGS. 2 and 3, it was confirmed that the activities of HTRA1 and HTRA2 increased as the dose of E09 increased. This means that E09 activates HTRA1 and HTRA2 in a dose-dependent manner and also activates HTRA1 in human blood samples.

### Example 3. Confirmation of HTRA1 Activity According to Presence or Absence of E09 in HeLa Cells

To confirm whether HTRA1 activity increases even when cells are treated with E09, HTRA1 activity according to the presence or absence of E09 was identified by treating Hela cells, which are human adenocarcinoma cells, with an HTRA1 activity-based probe (DS-1-94, final concentration of 5 μM) and E09 (HTRA activator, final concentration of 10 μM).

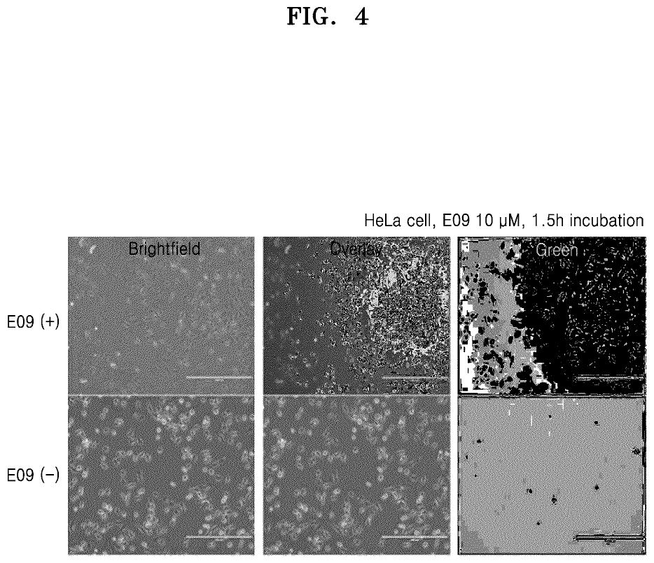

Specifically, the cells treated with the HIRAI activity-based probe (DS-1-94) and E09 (HTRA activator) were maintained in a 37 °C incubator for 1 hour. Thereafter, the cells were observed with a fluorescence microscope to identify how the probe reacts in living cells, and the results are shown in FIG. 4.

FIG. 4 is a photograph of HTRA1 activity observed with a fluorescence microscope after treating HeLa cells with an HTRA1 activity-based probe (DS-1-94, final concentration of 5 μM) and E09 (HTRA activator, final concentration of 10 μM).

As shown in FIG. 4, it was confirmed that a fluorescence signal markedly increased in the cells treated with the activator (E09). This means that HTRA1 activity is increased when the cells are treated with E09.

### Example 4. Measurement of Changes in HTRA Activity According to Addition of HTRA Activator

### 4.1 Preparation of HTRA Activity-Based Probes

HTRA activity-based probes were manufactured by a method disclosed in the prior registered patent KR 10-2302971 of the present inventor.

Specifically, P1-V tetra-peptide probe series (IRRV-probe, FIMV-probe, FKLV-probe, FKGV-probe, ASP-probe, LFLV-probe, MTP-LFLV-probe, and TPP-LFLV-probe) and P1-L tetra-peptide probe series (FIML-probe, FKLL-probe, FKGL-probe, ASPL-probe, and LFLL-probe) were produced by selecting hydrophobic functional groups of Val and Leu as amino acid functional groups located at P1 of a substrate-binding domain (or an active domain) of HTRA, which is well-conserved among HTRA proteins. In addition, to identify sub-family selectivity, each series was designed by a method of changing various types of amino acid functional groups at the P2 position, P3 position, and P4 position of the P1-V tetra-peptide probe series and the P1-L tetra-peptide probe series. Thirteen types of tetra-peptide probes were synthesized based on HTRA substrate amino acid sequences reported through prior research results.

### 4.2 Comparison of HTRA Enzyme Activity by HTRA Activator

HTRA activity was measured utilizing E09 and the HTRA activity-based probes synthesized by the method of Example 4.1. Changes in HTRA activity w ere measured for cases in which casein, known as a substrate of an HTRA enzyme, or PDZ-opt (GQYYFV), known as a peptide that activates an HTRA enzyme, was added and a case in which E09 was added.

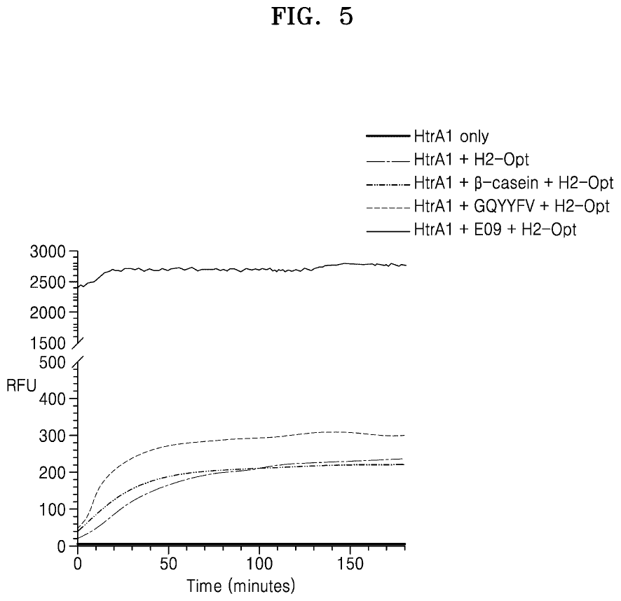

Specifically, 2 μg of β-casein or PDZ-opt (GQYYFV) or 10 μM of E09 was added to an HTRA1 enzyme by the method of Example 1, the mixture was allowed to stand at room temperature for 15 minutes, and then HTRA1 activity was measured at 37 °C for 3 hours in the same manner as in Example 1, and the results are shown in FIG. 5.

FIG. 5 is a graph showing HTRA1 enzyme activity measured using casein known as a substrate of HTRA enzymes, or PDZ-opt (GQYYFV), known as a peptide that activates HTRA enzymes, and E09.

As shown in FIG. 5, it was confirmed that E09 activates the HTRA1 enzyme at room temperature far more significantly than casein or PDZ-opt (GQYYFV).

These results indicate that the utilization of E09 enables the effective measurement of HTRA activity even with a low dose of an HTRA enzyme without a temperature change or the addition of a substrate.

### Example 5. Confirmation of the Effect of E09 in a NaIO₃-Induced Macular Degeneration Cell Model

In order to confirm the preventive or therapeutic effect of compound E09 on age-related macular degeneration (AMD), a dry age-related macular degeneration (AMD)-like cell model induced by treatment with NaIO₃ was established.

Specifically, ARPE-19 retinal pigment epithelial cells were seeded at a density of 1.66× 10⁵ cells/cm² and cultured for two weeks in a medium containing nicotinamide. Thereafter, the cells were re-seeded onto laminin-coated Transwell inserts.

After culture for one month in a medium containing nicotinamide, the cells were treated with NaIO₃ for 12 hours to induce cell injury (NaIO₃-alone treatment group), wherein NaIO₃ is commonly used for inducing dry age-related macular degeneration (AMD). Structural changes in the cells were then evaluated by analyzing clusterin and ZO-1. E09 and the HtrA1 inhibitor were dissolved in dimethyl sulfoxide (DMSO) and used for the experiments.

In order to evaluate the effect of E09 on NaIO₃-induced cell injury, E09 was pretreated 24 hours prior to NaIO₃ treatment and subsequently co-treated with NaIO₃ for 12 hours (E09-treated group).

In addition, under the same conditions, NaIO₃ and an HtrA1 inhibitor were administered, and a TUNEL assay was performed to evaluate cytotoxicity.

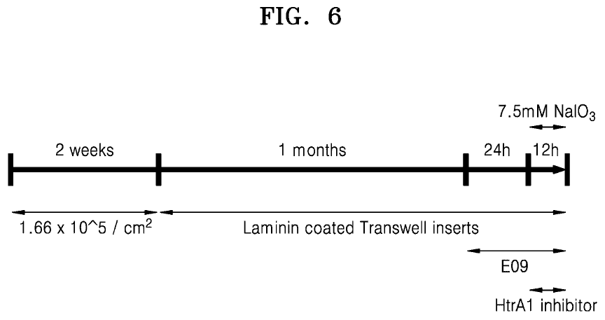

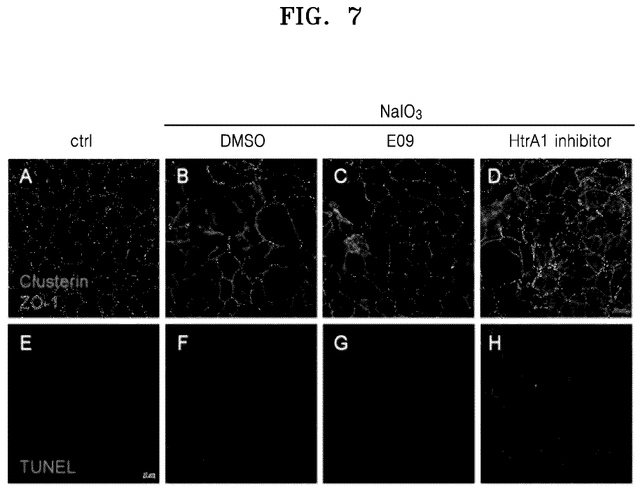

FIG. 6 is a schematic diagram showing a construction process of a dry macular degeneration cell model by NaIO₃ treatment and experimental conditions for each treatment group.

FIG. 7 is a set of images identifying changes in a cobblestone appearance structure according to NaIO₃ treatment through clusterin and ZO-1 staining (FIGS. 7A-D) and cell death confirmed by a TUNEL assay (FIGS. 7E-H).

In FIGS. 7E-7H, red dots indicate apoptotic cells (ctrl: untreated group without NaIO₃ treatment; DMSO: NaIO₃-alone treatment group; E09: group pretreated with E09 prior to NaIO₃ treatment and subsequently co-treated with NaIO₃ and E09 for 12 hours; HtrA1 inhibitor: group treated with an HtrA1 inhibitor under the same conditions as the E09-treated group).

As shown in FIG. 7, treatment with NaIO₃ markedly disrupted the normal cobblestone appearance structure (FIG. 7B). In contrast, in the group pretreated with E09, structural damage induced by NaIO₃ was significantly suppressed, and the monolayer structure of the cells was preserved (FIG. 7C). By comparison, in the group treated with the HtrA1 inhibitor, the cobblestone structure was irregularly disrupted (FIG. 7D).

In addition, according to the results of the TUNEL assay, cell death was markedly increased in the NaIO₃-alone treatment group (DMSO) (FIG. 7F), whereas NaIO₃-induced cell death was significantly suppressed in the E09-treated group (FIG. 7G). In contrast, in the HtrA1 inhibitor-treated group, a tendency toward increased cell death was observed (FIG. 7H).

These results indicate that E09 effectively suppresses structural damage and cell death of retinal pigment epithelial cells induced by treatment with NaIO₃, suggesting that E09 may be usefully employed for the prevention or treatment of dry age-related macular degeneration (AMD).

### Example 6. Confirmation of the Effect of E09 in a Nicotinamide-Induced Three-Dimensional Long-Term Culture Macular Degeneration Cell Model

In order to verify the preventive or therapeutic effect of compound E09 on age-related macular degeneration (AMD) under conditions similar to an in vivo environment, a macular degeneration cell model using a three-dimensional (3D) long-term culture system was established.

The model was designed to induce apical-basal polarization of ARPE-19 cells and to reproduce the structural and functional differentiation characteristics of retinal pigment epithelium (RPE), as well as pathological drusen accumulation patterns, through nicotinamide-based long-term culture.

Specifically, ARPE-19 retinal pigment epithelial cells were seeded at a density of 1.66× 10⁵ cells/cm² and cultured for two weeks in a medium containing nicotinamide. Thereafter, the cells were re-seeded onto laminin-coated Transwell inserts.

Subsequently, the medium was replaced every 2-3 days with a medium containing E09 or an HtrA1 inhibitor, and the cells were differentiated for 4.5 months (untreated control group (ctrl), E09-treated group, and HtrA1 inhibitor-treated group).

After long-term culture for 4.5 months to induce structural and functional differentiation of retinal pigment epithelium (RPE) comparable to in vivo conditions, immunostaining and morphological analyses were performed to evaluate the pathological and differentiation status of the cells.

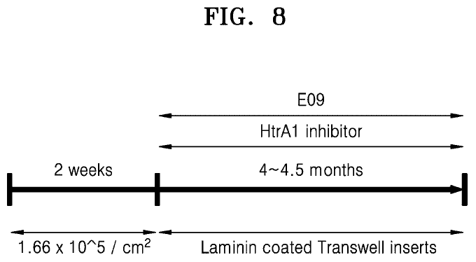

FIG. 8 is a schematic diagram illustrating a construction process of a three-dimensional long-term culture macular degeneration cell model and experimental conditions for each treatment group.

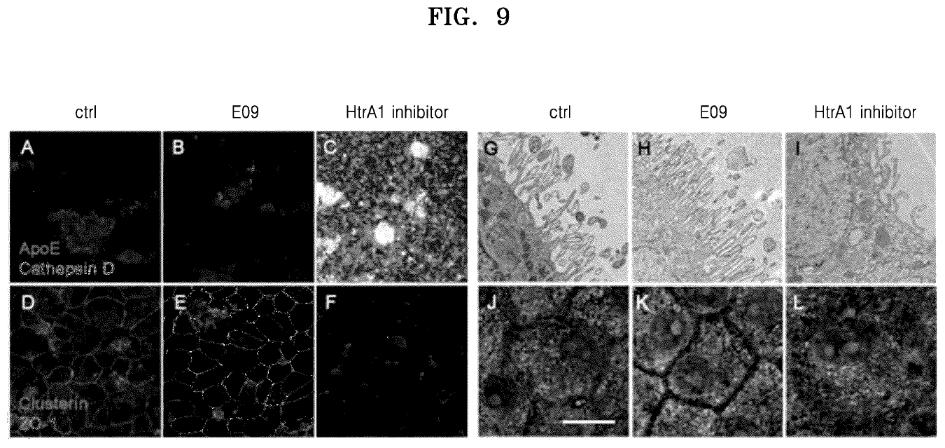

FIG. 9 is a set of images identifying expression levels of ApoE and Cathepsin D confirmed by immunostaining (FIGS. 9A-C), changes in a cell barrier structure according to clusterin and ZO-1 staining (FIGS. 9D-F), results of observing microvilli formation (FIGS. 9G-D), and results of holotomography analysis (FIGS. 9J-L).

As shown in FIG. 9, immunostaining analysis of ApoE and cathepsin D, which are known drusen markers accumulated in macular degeneration, demonstrated that, in the E09-treated group, expression levels of these markers tended to decrease compared with the control group, whereas in the HtrA1 inhibitor-treated group, ApoB and cathepsin D were markedly accumulated within the cells (FIGS. 9A-9C).

In addition, according to the staining results for ZO-1 and clusterin, which are markers of epithelial barrier differentiation, the E09-treated group maintained a continuous and regular staining pattern along the cell boundaries, indicating formation of a robust epithelial barrier structure. In contrast, in the HtrA1 inhibitor-treated group, continuity of the staining pattern was reduced and structural instability was observed (FIGS. 9D-9F).

Meanwhile, holotomography analysis further confirmed that a stable cytoskeletal structure was maintained in the E09-treated group, whereas structural instability was observed in the HtrA1 inhibitor-treated group (FIGS. 9J-9L). Analysis of microvilli formation, which is an important indicator of normal RPE function, demonstrated increased microvilli development in the E09-treated group, whereas formation tended to be suppressed in the HtrA1 inhibitor-treated group (FIGS. 9G-9I).

These results indicate that E09 maintains functional maturation and structural stability of retinal pigment epithelium (RPE), and suppresses drusen accumulation and epithelial barrier disruption, which are major pathological features of macular degeneration. This suggests that E09 may be usefully employed for the prevention or treatment of dry age-related macular degeneration (AMD).

### Example 7. Confirmation of the Effect of E09 in a NaIO₃-Induced Mouse Macular Degeneration Model

In order to verify the preventive or therapeutic effect of compound E09 on macular degeneration in an actual in vivo environment, a mouse macular degeneration pathology model induced by administration of NaIO₃ was established.

Specifically, C57BL/GJ mice (6 weeks old) were acclimated for one week, and on experimental day 0, NaIO₃ was administered via intravenous injection (IV) to induce retinal injury. To evaluate drug efficacy, E09 or an HtrA1 inhibitor was pre-administered by intravitreal injection one day prior to NaIO₃ administration (Day −1).

Five days after administration of NaIO₃, the eyes were enucleated, and the tissues. were fixed and sectioned. Hematoxylin & Eosin (H&E) staining was then performed to observe morphological changes in the retina.

In particular, the structural preservation status and thickness changes of the retinal pigment epithelium (RPE) layer, photoreceptor outer segments (POS), and the outer nuclear layer (ONL) were primarily analyzed.

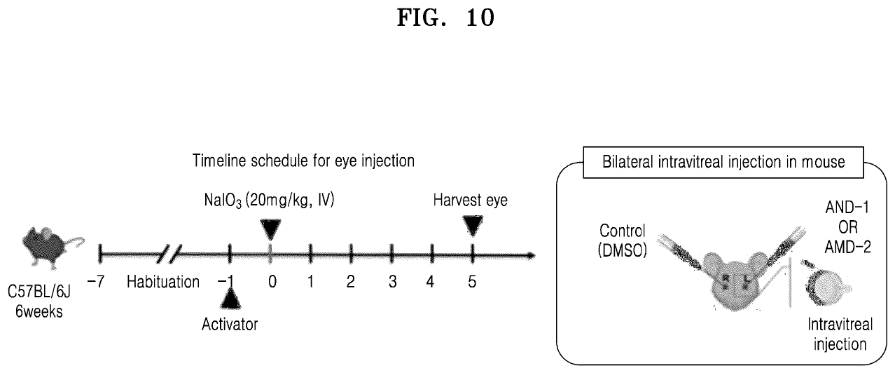

FIG. 10 is a schematic diagram illustrating the process for constructing a mouse macular degeneration pathology model induced by administration of NaIO₃.

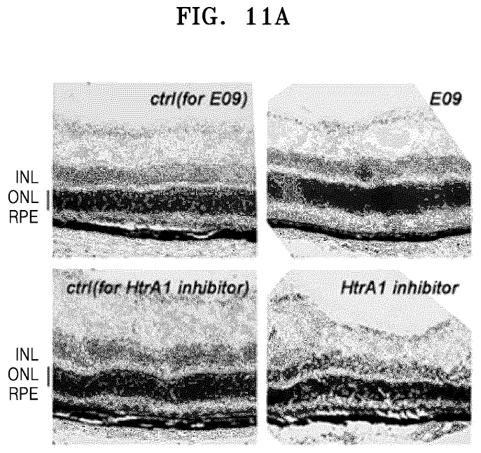

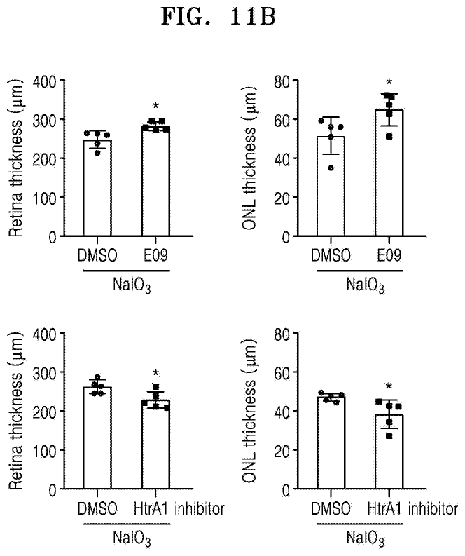

FIG. 11 shows results of comparative analysis on retinal structure protective effects of compound E09 and an HtrA1 inhibitor in a mouse macular degeneration pathology model induced by NaIO₃ administration:

FIG. 11A is a set of histological images observing morphological changes in a retinal pigment epithelium (RPE) layer, photoreceptor outer segments (POS), and an outer nuclear layer (ONL) by Hematoxylin & Eosin (H&E) staining of retinal tissues of a control group (DMSO), an E09-treated group, and an HtrA1 inhibitor-treated group after administration of NaIO₃; and FIG. 11B is statistical graphs comparing measurements of total retina thickness and ONL thickness to quantify the degree of retinal damage in each treatment group.

As shown in FIG. 11A, administration of NaIO₃ induced separation of the RPE and POS layers and severe structural damage in the control group (DMSO-treated group). However, in the group treated with compound E09, separation of the retinal layers was markedly suppressed, and tissue continuity was well maintained. In contrast, in the HtrA1 inhibitor-treated group, separation of the RPE-POS layers was further aggravated compared with the control group, indicating exacerbation of the pathological condition.

As shown in FIG. 11B, measurement of ONL thickness, which reflects photoreceptor cell density, demonstrated that the E09-treated group significantly suppressed NaIO₃-induced thinning and protected retinal structure. In contrast, in the HtrA1 inhibitor-treated group, ONL thickness was markedly reduced compared even to the corresponding control group (ctrl for HtrA1 inhibitor), indicating acceleration of retinal degeneration.

These results demonstrate that compound E09 effectively protects retinal pigment epithelium (RPE) and photoreceptor cells in a NaIO₃-induced dry age-related macular degeneration (AMD) model, thereby suppressing retinal degeneration.

In particular, the findings show that mere inhibition of HtrA1 activity may rather exacerbate the pathological condition, thereby indicating that compound E09 possesses a distinct and superior therapeutic mechanism and is effective for the prevention or treatment of dry age-related macular degeneration (AMD).

### Example 8. Elucidation of the Mechanism Underlying the Inhibitory Effect of Compound E09 on Macular Degeneration Pathology Through Transcriptome Analysis

In order to elucidate, at the molecular level, the mechanism of action of Compound E09, which was confirmed to exhibit a therapeutic effect in the pathological model of macular degeneration of Examples 5 to 7, a transcriptome analysis was performed. The collected transcriptome data were analyzed at the gene set level using Gene Set Enrichment Analysis (GSEA).

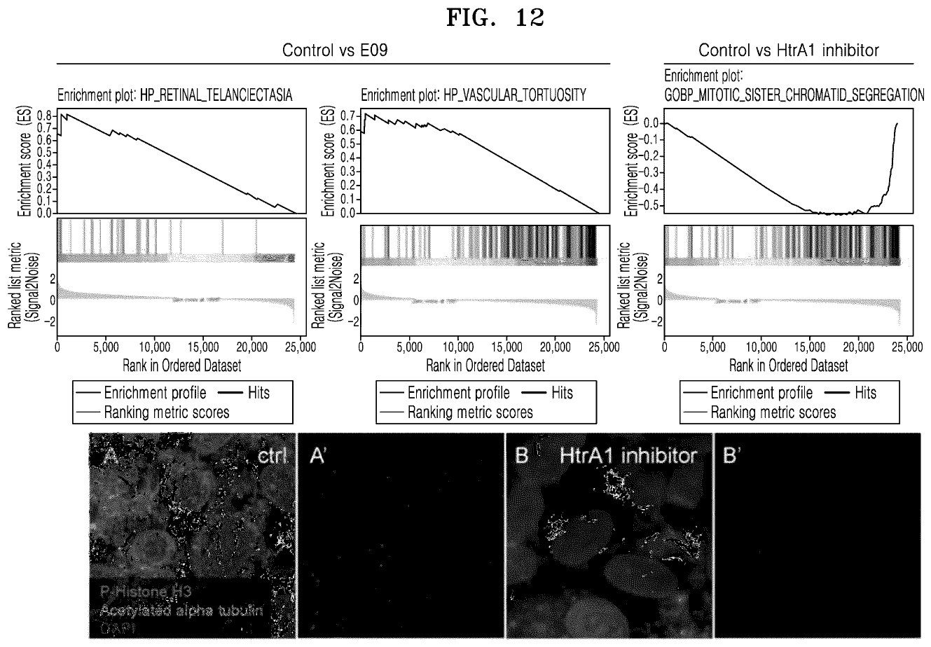

FIG. 12 is a graph showing the results of GSEA (Gene Set Enrichment Analysis) performed after transcriptome analysis following treatment with Compound E09 or an HTRA1 inhibitor in a pathological model of macular degeneration (AMD). The upper left graph of FIG. 12 shows the results of a comparison between the E09-treated group and a control group, and the upper right graph shows the results of a comparison between the HTRA1 inhibitor-treated group and the control group.

As shown in FIG. 12, in the case where Compound E09 was administered, as compared with the control group, gene sets associated with retinal vascular lesions, in particular gene sets related to retinal telangiectasia and vascular tortuosity, exhibited an overall tendency toward decreased expression (upper left graph of FIG. 12). This indicates that Compound E09 induces transcriptomic changes in a direction of suppressing pathological vascular dilation or abnormal vascular structural formation associated with macular degeneration.

In contrast, in the case where the HtrA1 inhibitor was administered, a pattern was confirmed in which gene sets associated with sister chromatid segregation and mitosis were significantly altered (upper right graph of FIG. 12). This suggests that, when the activity of HtrA1 is inhibited, abnormalities may be induced in normal cell cycle regulation and cell division processes.

These results indicate that Compound E09 exerts a therapeutic effect by selectively suppressing vascular abnormality-related transcriptional programs directly associated with macular degeneration pathology, whereas the HtrA1 inhibition strategy may cause abnormalities in cell cycle and mitotic regulation, thereby potentially disrupting cellular homeostasis.

Accordingly, the above results suggest that, rather than simply inhibiting the activity of HTRA1, a strategy for maintaining or enhancing the activity of HTRA1 may play a key role in normalizing the pathophysiology of macular degeneration.

1. A pharmaceutical composition for preventing or treating a degenerative disease, comprising: a compound of Formula 1, a stereoisomer thereof, a solvate thereof, a hydrate thereof, or a pharmaceutically acceptable salt thereof, as an active ingredient: 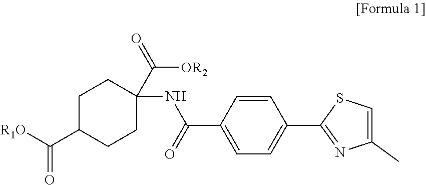
wherein, in Formula 1,
R₁ and R₂ are independently hydrogen, C₁-C₁₀ alkyl, C₂-C₁₀ alkenyl, or C₂-C₁₀ alkynyl.

2. The pharmaceutical composition of claim 1,
wherein the compound is one wherein R₁ and R₂ are independently hydrogen or C₁-C₄ alkyl, or a stereoisomer, solvate, hydrate, or pharmaceutically acceptable salt thereof.

3. The pharmaceutical composition of claim 1,
wherein the compound is one wherein R₁ and R₂ are independently hydrogen or methyl, or a stereoisomer, solvate, hydrate, or pharmaceutically acceptable salt thereof.

4. The pharmaceutical composition of claim 1, wherein the degenerative disease is age-related macular degeneration (AMD).

5. The pharmaceutical composition of claim 4, wherein the age-related macular degeneration (AMD) is one selected from the group consisting of wet macular degeneration, dry macular degeneration, geographic atrophy, and diabetic retinopathy.

6. The pharmaceutical composition of claim 1, wherein the degenerative disease is one selected from the group consisting of dementia, Alzheimer's disease (AD), Parkinson's disease, Huntington's disease, cerebral amyloid angiopathy, cerebral amyloid vasculopathy, amyloid-related stroke, systemic amyloidosis, Dutch-type amyloidosis, hereditary stroke (CADASIL), amyotrophic lateral sclerosis, spinocerebellar atrophy, Friedrich's ataxia, Machado-Joseph's disease, Lewy body dementia, dystonia, progressive supranuclear palsy, and frontotemporal dementia.

7. The pharmaceutical composition of claim 1, wherein the pharmaceutical composition activates a high temperature requirement A (HTRA).

8. The pharmaceutical composition of claim 7, wherein the HTRA is at least one selected from HTRA1 and HTRA2.

9-11. (canceled)

12. A method of screening a high temperature requirement A (HTRA) activity modulator, the method comprising:
activating HTRA by treating a sample comprising HTRA with a compound of Formula 1, a stereoisomer thereof, a solvate thereof, a hydrate thereof, or an acceptable salt thereof in vitro;
treating the sample comprising the activated HTRA with a candidate substance; and
comparing an HTRA activity level of the sample treated with the candidate substance with an HTRA activity level of a sample not treated with the candidate substance: 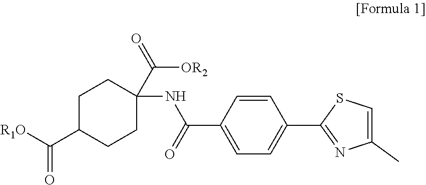
wherein, in Formula 1,
R₁ and R₂ are independently hydrogen, C₁-C₁₀ alkyl, C₂-C₁₀ alkenyl, or C₂-C₁₀ alkynyl.

13. The method of screening an HTRA activity modulator according to claim 12, wherein the comparing comprises selecting a candidate substance that has changed an HTRA activity level as an HTRA modulator.

14. The method of screening an HTRA activity modulator according to claim 12, wherein the sample is one selected from the group consisting of a cell, a tissue, blood, serum, urine, and saliva.

15. (canceled)

16. A method of preventing or treating a degenerative disease, the method comprising administering a composition comprising an effective amount of a compound of Formula 1, a stereoisomer thereof, a solvate thereof, a hydrate thereof, or a pharmaceutically acceptable salt thereof as an active ingredient to a subject in need thereof: 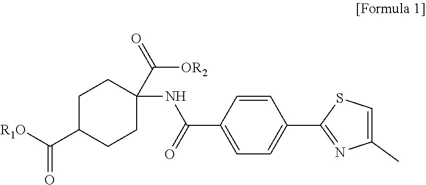
wherein, in Formula 1,
R₁ and R₂ are independently hydrogen, C₁-C₁₀ alkyl, C₂-C₁₀ alkenyl, or C₂-C₁₀ alkynyl.

17. (canceled)

18. The method of claim 16, wherein the degenerative disease is age-related macular degeneration (AMD).

19. The method of claim 18, wherein the age-related macular degeneration (AMD) is one selected from the group consisting of wet macular degeneration, dry macular degeneration, geographic atrophy, and diabetic retinopathy.
